(19) United States
(12) Patent Application Publication
(10) Pub. No.: US 2010/0029765 A1
(43) Pub. Date: Feb. 4, 2010

(54) TOPICAL AQUEOUS COMPOSITION COMPRISING TRETINOIN

(75) Inventors: Rahul Gupta, Jaipur (IN); Rajesh Pandurang Giradkar, Bhandara (IN)

Correspondence Address: Ranbaxy Inc., Intellectual Property Department, 600 College Road East, PRINCETON, NJ, 08540, US

(73) Assignee: RANBAXY LABORTORIES LIMITED, Gurgaon (IN)

(21) Appl. No.: 12/485,731

(22) Filed: Jun. 16, 2009

(30) Foreign Application Priority Data
Jul. 30, 2008 (IN) 1807/DEL/2008

(51) Int. Cl. A61K 31/203; A61P 17/10

(52) U.S. Cl. 514/559

(57) ABSTRACT

Topical aqueous compositions for the treatment of a skin disorder particularly acne. Topical aqueous composition comprising tretinoin and a hydrophilic cellulose derivative as a gelling agent, wherein the composition has a pH of about 4 to about 6.5 and viscosity of less than about 20,000 cP or provided. The composition also relates to the topical administration of tretinoin in combination with an antibiotic.

### TECHNICAL FIELD OF THE INVENTION

The present invention relates to topical aqueous compositions comprising tretinoin and a hydrophilic cellulose derivative as a gelling agent for the treatment of acne. The compositions also relate to the topical administration of tretinoin in combination with an antibiotic.

### BACKGROUND OF THE INVENTION

Skin disorders involving the sebaceous glands and follicles in humans include conditions such as acne and rosacea, as well as other noninfectious dermatological diseases involving microorganisms. Such disorders are often marked by inflammation.

Acne vulgaris or Acne is a common skin disorder characterized by blackheads, whiteheads, papules, pustules, cysts, and various sized nodules and scars, which in the inflammatory state of the disorder, are contaminated with bacteria such as Propionibacterium acnes. This disorder affects skin areas where the sebaceous glands are most active. Acne is most common during adolescence affecting more than 85% of teenagers, and frequently continues into adulthood.

Therapeutic methods for treating acne include the systemic and topical administration of anti-acne agents such as antibiotics or derivatives of Vitamin A acid but the topical treatment is preferred because it minimizes any potential systemic adverse effects and it is also less expensive.

Topical agents for the treatment of acne include retinoids like tretinoin and adapalene; sulfur; resorcinol; salicylic acid; benzoyl peroxide and antibiotics like erythromycin, clindamycin or tetracyclines.

Antimicrobial resistance to topical therapy is becoming an important factor in the treatment of acne, and clinically an association between the presence of antimicrobial resistant organisms and therapeutic failure has been made. The concomitant administration of two or more antiacne agents prevents the development of resistant microorganisms and proves to be more effective in the treatment of acne.

For example, one currently available combination product is Benzamycin topical gel (Dermik Laboratories, Berwyn, Pa.), which contains 3% of erythromycin and 5% of benzoyl peroxide. Another combination product marketed for the treatment of acne is Benzaclin topical gel (Sanofi Aventis), which contains 1% of clindamycin as phosphate and 5% of benzoyl peroxide.

Further, the combination of antibiotics and retinoids shows synergism in the treatment of acne. Both these agents act through different mechanisms thereby providing a synergistic action. Antibiotics prevent the growth of bacteria such as Propionibacterium acnes and retinoids have a keratolytic action and they also decrease the cohesiveness of follicular epithelial cells with decreased microcomedo formation.

A common retinoid being used in the treatment of acne is tretinoin. Tretinoin, also known as all-trans retinoic acid or Vitamin A acid is derived from Vitamin A by two oxidative steps. It is unstable and degrades in the presence of large amount of water. It is more susceptible to oxidation and decomposition when present in an aqueous medium. Therefore topical compositions of tretinoin have been formulated in non-aqueous vehicles. For example a cream formulation of tretinoin is presently approved and is commercially available from Ortho Pharmaceutical Company under the trademark RETIN-A. These non-aqueous compositions tend to irritate and dry the skin if applied frequently. The use of water-based preparation on the other hand would allow for maintenance of normal skin turgor and consistency by providing a moisturizing action.

Therefore, various approaches have been tried to formulate stable aqueous gel preparations of tretinoin. For example, one such approach is RETIN-A micro gel. This gel is being marketed by Advanced Polymer Systems and is, associated with U.S. Pat. No. 5,955,109. This patent describes an aqueous gel of tretinoin in which porous polymeric microbead carriers are used to retain tretinoin. Further, U.S. Pat. No. 5,721,275 describes an aqueous gel of tretinoin in which high molecular weight polyacrylate polymers have been used as a gelling agent. The Polyacrylic polymers also known as “Carbomers” are sensitive to electrolytes. Multivalent metal ions, in particular, cause a serious reduction in viscosity of the neutralized polymer. Their electrolytic sensitivity also compromises their application characteristics on the skin.

U.S. Pat. No. 5,670,547 describes a water-based formulation of tretinoin containing an acidic carboxy polymer as a gelling agent and a proteinaceous material which helps in stabilizing the gelling agent and it also provides humectant effects. The composition is said to be physically and chemically stable. However it is well known that proteinaceous materials are prone to microbial attack and chemical degradation especially in an aqueous vehicle. Therefore the use of proteinaceous material in the formulation leads to stability problems during storage and further increases the cost of the formulation.

Antibiotics commonly employed for the treatment of acne include lincomycin antibiotics for example clindamycin, macrolide antibiotics for example erythromycin or tetracyclines for example minocycline. A common antibiotic used in the topical treatment of acne is clindamycin due to its ability to form stable compositions. Commercial products of clindamycin for the treatment of acne include Cleocin T solution, gel and lotion marketed by Pharmacia and Upjohn. Compositions containing clindamycin are disclosed in U.S. Pat. No. 3,969,516.

### SUMMARY OF THE INVENTION

There exists a need for an aqueous composition of tretinoin particularly in combination with an antibiotic and a hydrophilic gelling agent other than the electrolytic sensitive carbomers.

The inventors have presently developed an aqueous composition for topical administration of tretinoin comprising a hydrophilic cellulose derivative as a gelling agent. Particularly the gelling agents are hydroxyethylcellulose and sodium carboxy methylcellulose. Hydroxyethylcellulose dissolves readily in water to give clear, smooth, viscous solutions that are non-toxic. The solutions prepared with hydroxyethyl cellulose are less affected by pH change and are more tolerant of the presence of anions. The stiffness of sodiumcarboxymethyl cellulose based gel increases with increase in its concentration and molecular weight. The composition can also be utilized for the topical administration of tretinoin in combination with an antibiotic.

Disclosed herein is a topical aqueous composition of tretinoin, particularly in a combination with an antibiotic.

In one aspect there is provided a topical aqueous composition comprising:
(a) a therapeutically effective amount of tretinoin; and
(b) a hydrophilic cellulose derivative as a gelling agent,
   wherein, the composition has a pH of about 4 to about 6.5 and viscosity of less than about 20,000 cP.

According to one of the embodiments the hydrophilic cellulose derivative comprise one or more water-soluble cellulose ethers selected from, for example, methylcellulose, hydroxypropyl methylcellulose, hydroxyethyl cellulose, hydroxypropyl cellulose, carboxymethyl cellulose, sodium carboxymethyl cellulose and hydroxyethylmethyl cellulose. In some embodiments, the hydrophilic cellulose derivative is hydroxyethyl cellulose, sodium carboxymethyl cellulose, hydroxypropyl cellulose or mixtures thereof. According to one of the embodiments the hydrophilic cellulose derivative is hydroxyethyl cellulose.

According to another embodiment, tretinoin can be present in an amount of about 0.001% to about 0.5% by weight of the composition. In some embodiments, the composition can contain about 0.005% to about 0.05% by weight of tretinoin. For example, the composition can contain about 0.01% to about 0.025% by weight of tretinoin.

According to another embodiment hydrophilic cellulose derivative can comprise about 0.05% to about 30% of the total weight of the composition. For example, the composition can comprise about 1% to about 15% by weight of the total weight of the composition, or about 2% to about 10% by weight of hydrophilic cellulose derivative.

Another aspect relates to a method of preparing topical aqueous composition comprising tretinoin and a hydrophilic cellulose derivative as a gelling agent wherein the method comprises
(a) dispersing tretinoin in a water-miscible solvent water using a surfactant;
(b) dissolving preservative in purified water;
(c) dispersing gelling agent to form an aqueous solution or dispersion;
(d) combining (a), (b) & (c) to form a composition; and
(e) adjusting the pH to about 4 to about 6.5.

According to another aspect, there is provided a topical aqueous composition comprising:
(a) a therapeutically effective amount of tretinoin;
(b) a therapeutically effective amount of at least one antibiotic or pharmaceutically effective salts or esters thereof; and
(c) a hydrophilic cellulose derivative as a gelling agent,
   wherein, the composition has a pH of about 4 to about 6.5 and viscosity of less than about 20,000 cP.

According to one of the embodiments antibiotic is selected from, for example, lincomycins, erythromycins, tetracyclines or one of their derivatives thereof. Lincomycin derivatives include clindamycin, clindamycin phosphate, clindamycin hydrochloride or any other salt or ester thereof. Erythromycin derivatives include clarithromycin. Tetracycline derivatives include minocycline, meclocycline, doxycycline or any of their salts or esters thereof. In some embodimentsthe antibiotic used in compositions of present invention can be a lincomycin derivative, or clindamycin phosphate.

According to another embodiment, clindamycin phosphate can be present in an amount of about 0.1% to about 5.0% by weight of the composition. For example, the composition of present invention contains about 0.5% to about 2.0% by weight of clindamycin phosphate.

Another aspect relates to a method of preparing topical aqueous composition comprising a therapeutically effective amount of tretinoin and an antibiotic and a hydrophilic cellulose derivative as a gelling agent wherein the method comprises:
(a) dispersing tretinoin in a water-miscible solvent water using a surfactant;
(b) dissolving the antibiotic and preservative in purified water;
(c) dispersing gelling agent to form an aqueous solution or dispersion;
(d) combining (a), (b) & (c) to form a composition; and
(e) adjusting the pH to about 4 to about 6.5.

Another aspect provides a topical aqueous pharmaceutical composition wherein the said composition is in the form of a gel, solution, foam, lotion or spray. In some embodiments, the topical aqueous composition is in the form of a gel.

According to another aspect, a composition of one or more pharmaceutically acceptable excipients selected from, for example, water miscible solvents, preservatives, antioxidants, chelating agents, surfactants, pH-adjusting agents, fragrances, perfumes or mixtures thereof.

According to one of the embodiments, water miscible solvents can be, for example, the group comprising of ethanol, propylene glycol, glycerin, polyethylene glycol or mixtures thereof.

According to another embodiment, antioxidants can be, for example, butylated hydroxyanisole (BHA), butylated hydroxytoluene (BHT), sodium metabisulfite, ascorbic acid, ascorbyl palmitate, thiourea, acetylcysteine, dithiothreitol, cysteine hydrochloride, propyl gallate, or tocopherols.

According to another embodiment, preservatives can be, for example, methyl-, ethyl-, propyl- or butyl-esters of hydroxybenzoic acid, benzoic acid, chlorhexedine, benzalkonium chloride and 2-phenoxyethanol, cetrimide, potassium sorbate or thiomersal.

According to yet another embodiment, chelating agents can be, for example, edetate salts or citric acid.

According to still another embodiment, surfactants can be, for example, polyethoxylated fatty acid esters, polyoxyethylene sorbitan esters, polyoxyethylene hydrogenated castor oil, polyoxyethylene polyoxypropylene glycol, sorbitan esters, sodium lauryl sulphate, docusate sodium, nonooxynol and glyceryl monostearate.

According to another embodiment, pH adjusting agents can be, for example, sodium hydroxide, tromethamine or hydrochloric acid.

Another aspect provides a method of treating acne by administering a therapeutically effective amount of topical aqueous composition as described herein.

According to yet another aspect, additional antiacne agents can be included in particular compositions. Examples of additional anti-acne agents may include, but are not limited to, benzoyl peroxide, salicylic acid, azelaic acid, retinoids other than tretinoin, metronidazole or mixtures thereof.

### DESCRIPTION OF THE INVENTION

Topical aqueous compositions for the treatment of a skin disorder particularly acne are provided. The topical compositions comprise a therapeutically effective amount of tretinoin and a hydrophilic cellulose derivative having gelling properties and capable of providing a constant and uniform release of active pharmaceutical ingredients. Compositions may also comprise an antibiotic in combination with tretinoin.

The phrase “therapeutically effective amount” as used herein means the amount of a compound that, when administered to a subject for treating a state, disorder, condition or causing an action is sufficient to effect such treatment or action. The “therapeutically effective amount” will vary depending on the compound, the disease and its severity and the age, weight, physical condition and responsiveness of the mammal to be treated.

Tretinoin is all-trans retinoic acid or Vitamin A acid. Chemically, tretinoin is 3,7-dimethyl-9-(2,6,6-trimethyl-1-cyclohexen-1-yl)-2,4,6,8-nonatetraenoic acid. Tretinoin may be present in an amount of about 0.001% to about 0.5% by weight of the composition. For example, tretinoin can be present in an amount of about 0.005% to about 0.05% by weight of the composition, or for example, about 0.01 to about 0.025% by weight of the composition.

An aqueous gel composition for topical administration of tretinoin to the skin is provided, which increases the therapeutic effectiveness of such an application over alcoholic gel vehicles or oil-based vehicles while reducing the irritation that can be associated with the application of tretinoin to the skin of certain sensitive patients.

The gelling agent can be is a hydrophilic cellulose derivative. As used herein the phrase “hydrophilic cellulose derivative” includes water soluble cellulose ethers, for example methyl cellulose, hydroxypropyl methylcellulose, hydroxyethyl cellulose, hydroxypropyl cellulose, carboxymethyl cellulose, sodium carboxymethyl cellulose and hydroxyethylmethyl cellulose. In particular embodiments, the gelling agents can be hydroxyethylcellulose (HEC) and sodium carboxy methylcellulose. Hydroxyethyl cellulose is available under the trade name NATROSOL®. Medium or high viscosity grades of NATROSOL® are used to control rheology, provide thickening and pseudoplasticity to gels. Grades 250 M, H, HX and HHX are typically chosen for topical gel formulations.

Sodium carboxymethyl cellulose is available under three different viscosity grades: low, medium and high. The stiffness of sodium carboxymethyl cellulose-based gel increases with increase in its concentration and molecular weight. The gelling agent may be present in an amount of about 0.05% to about 30% of the composition, for example about 1% to about 15% by weight of the total weight of the composition, or for example about 2% to about 10% by weight of gelling agent.

The compositions may also contain an antibiotic in combination with tretinoin. Topical antibiotics used in the treatment acne include lincomycins, lincomycin derivatives, erythromycins, erythromycin derivatives, tetracyclines, tetracycline derivatives and their pharmaceutically acceptable salts, esters, or prodrugs thereof. Lincomycin derivatives include clindamycin, clindamycin phosphate, clindamycin hydrochloride or any other salt or ester thereof. Erythromycin derivatives include clarithromycin. Tetracycline derivatives include minocycline, meclocycline, doxycycline or any of their salts or esters thereof are used. In some embodiments clindamycin or pharmaceutically acceptable salts or esters thereof. Clindamycin is the 7-deoxy, 7-chloro derivative of lincomycin. Chemically clindamycin is described as methyl 7-chloro-6,7,8-trideoxy-6-(1-methyl-trans-4-propyl-L-2-pyrrolidinecarboxamido)-1-thio-L-threo-α.-D-galacto-octo-pyranoside.

As used herein the phrase “pharmaceutically acceptable salts or esters” of clindamycin include, but are not limited to, clindamycin hydrochloride, clindamycin phosphate, clindamycin palmitate, and clindamycin palmitate hydrochloride. In some embodiments, clindamycin phosphate is used.

The compositions can contain about 0.1% to about 5.0% by weight of clindamycin phosphate, for example, about 0.5% to about 2.0% by weight of clindamycin phosphate.

The pharmaceutical compositions can further include one or more pharmaceutically acceptable excipients, for example, water miscible solvents, antioxidants, preservatives, chelating agents, surfactants, pH-adjusting agents, fragrances, perfumes or mixtures thereof.

Suitable water-miscible solvents for use herein may include ethanol, propylene glycol, glycerin and polyethylene glycol. Certain water-miscible solvents, such as glycerin or propylene glycol also add beneficial humectant properties to the composition. The aqueous compositions may comprise up to 30% by weight of water-miscible solvent by total weight of the composition.

As the active ingredients may be susceptible to oxidation in an aqueous medium, an antioxidant can be used in the compositions to retard oxidation and deterioration of the active ingredients, thus providing the formulation with increased long-term stability. Specific examples of antioxidants include butylated hydroxyani sole (B HA), butylated hydroxytoluene (BHT), sodium metabisulfite, ascorbic acid, ascorbyl palmitate, thiourea, acetylcysteine, dithiothreitol, cysteine hydrochloride, propyl gallate, and the tocopherols. In some embodiments, the antioxidant can be butylated hydroxytoluene (BHT). Antioxidants may be present in an amount of about 0.01% to about 0.3% by weight of the composition.

The compositions may comprise about 0.005% to about 2.0% by weight of preservatives by total weight of the composition. Examples of preservatives include methyl-, ethyl-, propyl- and butyl-esters of hydroxy benzoic acid, benzoic acid, chlorhexedine, benzalkonium chloride and 2-phenoxyethanol, cetrimide, potassium sorbate and thiomersal.

Suitable chelating agents include edetate salts for example edetate disodium and citric acid. Chelating agents chelate metal ions present in the composition that may be detrimental to the shelf life of the formulation. In some embodiments the chelating agent is present in an amount of from 0.01 to 0.5% by weight by total weight of the composition.

A surfactant may also be included in the formulations to allow good dispersion of the active ingredients. Examples of surfactants include polyethoxylated fatty acid esters, polyoxyethylene sorbitan esters, polyoxyethylene hydrogenated castor oil, polyoxyethylene polyoxypropylene glycol, sorbitan esters, sodium lauryl sulphate, docusate sodium, nonooxynol and glyceryl monostearate. The aqueous gel composition of the present invention can comprise from about 0.001% to about 5.0% surfactant weight by total weight of the composition.

The pharmaceutical compositions can also contain one or more pH-adjusting agents. Useful pH-adjusting agents include pharmaceutically acceptable organic or inorganic acids or bases; for example sodium hydroxide, tromethamine and hydrochloric acid. In some embodiments the compositions of the present invention have a pH of about 4 to about 6.5.

Examples of fragrances and perfumes include Lavender oil, Rose oil, Lemon oil, Almond oil.

The compositions may be present in the form of a gel, solution, foam, lotion or spray for topical application. In some embodiments, the compositions are in the form of an aqueous gel.

The viscosity of the compositions be less than about 20,000 cP, for example, between about 100 and about 15,000 cP, or for example between about 500 and about 10,000 cP. The viscosity is determined at room temperature (20-25° C.) using a Brookfield viscometer model RVT, spindle #2 at 20 revolutions per minute (rpm).

To prepare an aqueous gel with two active ingredients where one is suspended and the other is dissolved, an insoluble active can be added to a water-miscible ingredient, or a portion of the water with a surfactant, to disperse. Separately, another active and any other preservative ingredients can be dissolved in the purified water. The gelling agent can be dispersed in the aqueous solution with appropriate stirring. Then the dispersion of the first active ingredient can be added to the gel and mixed well to blend. Lastly, a pH-adjusting agent can be added to adjust the pH to the desired range. Aqueous gel can be formed by using hydrophilic cellulose derivative as gelling agent, with the clindamycin phosphate dissolved and the tretinoin suspended.

Methods for treating a skin disorder, particularly acne in a human, are provided which method comprises administering a composition to an affected area of the subject's skin having such disorder in an amount and for a period of time sufficient to improve the skin disorder. In some embodiments, the composition is administered once a day over the treatment period. Depending on the patient's improvement, the treatment may extend for less than a week to two months or more. The progress of improvement may be monitored by the patient or by a physician.

Additional antiacne agents may also be included in the compositions of the present invention. Examples of additional anti-acne agents include but are not limited to benzoyl peroxide, salicylic acid, azelaic acid, retinoids other than tretinoin, metronidazole or mixtures thereof.

The following examples can be used to illustrate the invention, but do not limit the scope of invention.

### EXAMPLE 1

### Aqueous Gel Composition Comprising Tretinoin

| S. | | Percent (%) w/w |
|---|---|---|
| No. | Ingredients | (total weight of the dosage form) |
| 1 | Tretinoin | 0.025 |
| 2 | Glycerin | 10.000 |
| 3 | Hydroxyethyl cellulose | 2.200 |
| 4 | Methylparaben | 0.180 |
| 5 | Polysorbate 80 | 2.000 |
| 6 | Edetate disodium | 0.100 |
| 7 | Citric acid | 0.050 |
| 8 | Propylparaben | 0.020 |
| 9 | Butylated hydroxytoluene | 0.075 |
| 10 | Tromethamine q.s to adjust pH | q.s. |
| 11 | Purified water | q.s. to 100.000 |

### Process:

1 Methyl paraben and propyl paraben were added to hot water (70-90° C.) and the solution was allowed to cool below 30° C.
2 Disodium edetate and citric acid were dissolved in the solution of step 1.
3 Tretinoin and butylated hydroxytoluene were dissolved in polysorbate 80 with stirring.
4. Glycerin was added to step 3 mixture with stirring.
5. Mixture of step 4 was added to the solution of step 2 with stirring.
6. Hydroxyethyl cellulose was added to step 5 with stirring.
7. The pH was adjusted with 5% Tromethamine solution.
8. Volume was made up to the batch size by adding water and mixture was stirred to get a uniform solution.
9. The gel was packed in Aluminium or collapsible tubes.

### EXAMPLE 2

### Aqueous Gel Composition Comprising Tretinoin and Clindamycin Phosphate

| S. | | Percent (%) w/w |
|---|---|---|
| No. | Ingredients | (total weight of the dosage form) |
| 1 | Clindamycin phosphate | 1.200 |
| 2 | Tretinoin | 0.025 |
| 3 | Glycerin | 10.000 |
| 4 | Hydroxyethyl cellulose | 2.200 |
| 5 | Methylparaben | 0.180 |
| 6 | Polysorbate 80 | 2.000 |
| 7 | Edetate disodium | 0.100 |
| 8 | Citric acid | 0.050 |
| 9 | Propylparaben | 0.020 |
| 10 | Butylated hydroxytoluene | 0.075 |
| 11 | Tromethamine q.s to adjust pH | q.s. |
| 12 | Purified water | q.s. to 100.000 |

### Process:

1. Methyl paraben and propyl paraben were added to hot water (70-90° C.) and the solution was allowed to cool below 30° C.
2. Disodium edetate, citric acid and Clindamycin phosphate were dissolved in the solution of step 1.
3. Tretinoin and butylated hydroxytoluene were dissolved in polysorbate 80 with stirring.
4. Glycerin was added to step 3 mixture with stirring.
5. Mixture of step 4 was added to the solution of step 2 with stirring.
6. Hydroxyethyl cellulose was added to step 5 with stirring.
7. The pH was adjusted with 5% Tromethamine solution.
8. Volume was made up to the batch size by adding water and mixture was stirred to get a uniform solution.
9. The gel was packed in Aluminium or collapsible tubes.

### EXAMPLE 3

### Aqueous Gel Composition Comprising Tretinoin and Clindamycin Phosphate

| S. | | Percent (%) w/w |
|---|---|---|
| No. | Ingredients | (total weight of the dosage form) |
| 1 | Clindamycin phosphate | 1.200 |
| 2 | Tretinoin | 0.025 |
| 3 | Glycerin | 10.000 |
| 4 | Sodium carboxymethyl cellulose | 2.200 |
| 5 | Methylparaben | 0.180 |
| 6 | Polysorbate 80 | 2.000 |
| 7 | Edetate disodium | 0.100 |
| 8 | Citric acid | 0.050 |
| 9 | Propylparaben | 0.020 |
| 10 | Butylated hydroxytoluene | 0.075 |
| 11 | Tromethamine q.s to adjust pH | q.s. |
| 12 | Purified water | q.s. to 100.000 |

### Process:

1. Methyl paraben and propyl paraben were added to hot water (70-90° C.) and the solution was allowed to cool below 30° C.
2. Disodium edetate, citric acid and Clindamycin phosphate were dissolved in the solution of step 1.
3. Tretinoin and butylated hydroxytoluene were dissolved in polysorbate 80 with stirring.
4. Glycerin was added to step 3 mixture with stirring.
5. Mixture of step 4 was added to the solution of step 2 with stirring.
6. Sodium carboxymethyl cellulose was added to step 5 with stirring.
7. The pH was adjusted with 5% Tromethamine solution.
8. Volume was made up to the batch size by adding water and mixture was stirred to get a uniform solution.
9. The gel was packed in Aluminium or collapsible tubes.

While several particular forms of the invention have been illustrated and described, it will be apparent that various modifications and combinations of the invention detailed in the text can be made without departing from the spirit and scope of the invention.

We claim:

1. A topical aqueous composition comprising a therapeutically effective amount of tretinoin and a hydrophilic cellulose derivative as a gelling agent, wherein the composition has a pH of about 4 to about 6.5 and viscosity of less than about 20,000 cP.

2. The topical aqueous composition according to claim 1 wherein the hydrophilic cellulose derivative is selected methyl cellulose, hydroxypropyl methylcellulose, hydroxyethyl cellulose, hydroxypropyl cellulose, carboxymethyl cellulose, sodium carboxymethyl cellulose and hydroxyethylmethyl cellulose and mixtures thereof.

3. The topical aqueous composition according to claim 1 wherein the hydrophilic cellulose derivative is present in an amount of about 1% to about 15% by weight of the composition.

4. The topical aqueous composition according to claim 1 wherein tretinoin is present in an amount of about 0.001% to about 0.5% by weight of the composition.

5. A method of preparing a topical aqueous composition according to claim 1, the method comprising:
a. dispersing tretinoin in a water-miscible solvent water using a surfactant;
b. dissolving preservative in purified water;
c. dispersing a gelling agent to form an aqueous solution or dispersion;
d. combining (a), (b) & (c) to form a composition; and
e. adjusting the pH to about 4 to about 6.5.

6. A topical aqueous composition comprising:
(a) a therapeutically effective amount of tretinoin;
(b) a therapeutically effective amount at least one antibiotic or salts or esters thereof; and
(c) a hydrophilic cellulose derivative as a gelling agent, wherein the composition has a pH of about 4 to about 6.5 and viscosity of less than about 20,000 cP.

7. The topical aqueous composition according to claim 6 wherein the antibiotic is selected from lincomycins, lincomycin derivatives, erythromycin, erythromycin derivatives, tetracycline, tetracycline derivatives and their pharmaceutically acceptable salts, esters, or prodrugs thereof.

8. The topical aqueous composition according to claim 7 wherein the antibiotic is clindamycin or pharmaceutically acceptable salts or esters thereof.

9. The topical composition according to claim 8 wherein the clindamycin salts or esters are selected from clindamycin hydrochloride, clindamycin phosphate, clindamycin palmitate, or clindamycin palmitate hydrochloride.

10. The topical aqueous composition according to claim 9 wherein clindamycin phosphate is present in an amount of about 0.1% to about 5% by weight of the composition.

11. A method of preparing a topical aqueous composition according to claim 6, the method comprising:
a. dispersing tretinoin in a water-miscible solvent water using a surfactant;
b. dissolving an antibiotic and preservative in purified water;
c. dispersing a gelling agent to form an aqueous solution or dispersion;
d. combining (a), (b) & (c) to form a composition; and
e. adjusting the pH to about 4 to about 6.5.

12. The topical aqueous composition according to claim 1 further comprising one or more pharmaceutically acceptable excipients selected from one or more of water-miscible solvents, antioxidants, preservatives, chelating agents, surfactants, pH-adjusting agents, fragrances, perfumes or mixtures thereof.

13. The topical aqueous composition according to claim 12 wherein the water-miscible solvents are selected from ethanol, propylene glycol, glycerin, polyethylene glycol or mixtures thereof.

14. The topical aqueous composition according to claim 12 wherein the antioxidants are selected from butylated hydroxyanisole (BHA), butylated hydroxytoluene (BHT), sodium metabisulfite, ascorbic acid, ascorbyl palmitate, thiourea, acetylcysteine, dithiothreitol, cysteine hydrochloride, propyl gallate, and tocopherols.

15. The topical aqueous composition according to claim 12 wherein the preservatives are selected from methyl, ethyl, propyl and butyl esters of hydroxy benzoic acid, benzoic acid, chlorhexedine, benzalkonium chloride and 2-phenoxyethanol, cetrimide, potassium sorbate and thiomersal.

16. The topical aqueous composition according to claim 12 wherein the chelating agents are selected from edetate salts or citric acid.

17. The topical aqueous composition according to claim 12 wherein the surfactants are selected from polyethoxylated fatty acid esters, polyoxyethylene sorbitan esters, polyoxyethylene hydrogenated castor oil, polyoxyethylene polyoxypropylene glycol, sorbitan esters, sodium lauryl sulphate, docusate sodium, nonooxynol and glyceryl monostearate.

18. The topical aqueous composition according to claim 12 wherein the pH-adjusting agents are selected from sodium hydroxide, tromethamine and hydrochloric acid.

19. The topical aqueous composition according to claim 1 wherein the composition is a gel, lotion, foam, solution or spray.

20. A method of treating acne in a patient by administering a therapeutically effective amount of the topical aqueous composition according to claim 1.
